(19) United States
(12) Patent Application Publication
(10) Pub. No.: US 2020/0000240 A1
(43) Pub. Date: Jan. 2, 2020

(54) SLEEP INDUCTION DEVICE

(71) Applicant: HFJ Life Business Co., Ltd., Saitama (JP)

(72) Inventors: Fujio HARA, Saitama (JP); Chun-Yuan HSIAO, Saitama (JP); Shao-Chi CHANG, Saitama (JP)

(21) Appl. No.: 16/488,934

(22) PCT Filed: Feb. 27, 2017
(86) PCT No.: PCT/JP2017/007422

§ 371 Date: Aug. 26, 2019

(51) Int. Cl. A47C 21/00; F16H 21/22; A61M 21/02; A61H 1/00

(52) U.S. Cl. CPC A47C 21/006; F16H 21/22; F16H 21/04; A61H 1/003; A61M 21/02

(57) ABSTRACT

A sleep induction device for oscillating a bed to induce a user of the bed to sleep can be applied to a bed of any size. A bed including a drive leg support including a movable leg holder slidable in a predetermined direction within a predetermined range, a drive means for reciprocally sliding the movable leg holder within a predetermined range, and a driven leg support including the movable leg holder, wherein the sleep induction device oscillates a bed having legs mounted on the drive leg support and the driven leg support by reciprocally oscillated by reciprocally sliding the movable leg holder of the drive leg support.

### TECHNICAL FIELD

The present invention relates to a sleep induction device for inducing a user to sleep by oscillating a bed.

### BACKGROUND ART

It is known that an environment with moderate oscillation causes relaxation of the human brain and spirit to produce a sleeping effect. For example, Non-Patent Document 1 discloses an experimental result in which a oscillation stimulus having a 1/f fluctuation characteristic is given to a subject so that the subject can sleep more quickly and deeply than in the case where the oscillation stimulus is not given. In addition, Non-Patent Document 2 discloses an experimental result that a phenomenon of getting sleepy when riding on a train tends to occur when the oscillation frequency of the train contains many low frequencies of about 1 Hz.

In the case of inducing a person to sleep comfortably by oscillating the bed, the method of generating oscillation includes a method of incorporating a oscillation mechanism in the bed itself (for example, see Patent Document 1), and a method of oscillating the bed by separating the bed from the oscillation mechanism and placing the bed on the oscillation mechanism (for example, see Patent Document 2).

### PRIOR ART REFERENCES

### Patent Document

Patent Document 1: Japanese Patent No. 2539989

Patent Document 2: Japanese Patent Application Laid-Open No. 2014-87457

### Non-patent document

Non-Patent Document 1: Jun Takamura, “Improved sleep penetration by 1/f fluctuation oscillation”, Textile Consumer Sciences, 1990, Vol. 31, No. 8, and p 365-370.

Non-Patent Document 2: Mitsuharu Shimoyama, “Why do you get sleepy on a train? The reason revealed in the research. Also use for good sleep” online, May 23, 2015, Vehicle News, [Search on Jan. 27, 2017], Internet (URL https://trafficnews.jp/post/40355/)

### SUMMARY OF THE INVENTION

### Problems to be Solved by the Invention

In the case of the method of Patent Document 1, since the oscillation mechanism is incorporated in the bed, the oscillation mechanism cannot be used for other beds. In the case of the method of Patent Document 2, although the bed and the oscillation mechanism are separated from each other, since the entire bed is mounted on the oscillation mechanism, the size of the mountable bed is limited by the size of the oscillation mechanism.

It is an object of the present invention to provide an asleep induction device which is a oscillation mechanism for oscillating a bed to induce a user of the bed to be asleep, and which can be applied to a bed of an arbitrary size.

### Means for Solving the Problems

(1) The sleep induction device of the present invention includes a drive leg support including a movable leg holder slidable within a predetermined range in a predetermined direction, a drive means for reciprocally sliding the movable leg holder within a predetermined range, and a driven leg support including the movable leg holder, and causes a bed on which leg portions are mounted on the drive leg support and the driven leg support to reciprocally oscillate by reciprocally sliding the movable leg holder of the drive leg support. This makes it possible to apply the sleep induction device to beds of any existing size.

(2) The direction of sliding may be the longitudinal direction of the bed, and the leg on one end side in the longitudinal direction of the bed may be mounted on the drive leg support, and the leg on the other end side may be mounted on the driven leg support. By setting the sliding direction to the longitudinal direction of the bed, it is possible to suppress irritation to the semicircular canal of the bed user in the recumbent position, and to induce the bed user to sleep comfortably.

(3) Since the frequency of the oscillation that leads the bed user to sleep is not necessarily constant, a control means for controlling the frequency of the reciprocating sliding of the drive means may be provided so that the oscillation can be appropriately controlled.

(4) The drive means may realize the reciprocating sliding by using a crank mechanism that converts the rotational movement by the motor into the reciprocating movement. In this case, the frequency of the reciprocating sliding can be controlled by controlling the rotation speed of the motor by the control means. The adoption of the crank mechanism creates an anomalous rhythm of decelerating, reversing, and accelerating before and after the turning portion of the reciprocating motion, and this rhythm can relax the user and lead the user to sleep comfortably.

(5) The control means may control the frequency of the reciprocating sliding to vary with time. Such an anomalous rhythm can also relax the user and lead the user to sleep comfortably.

(6) The control means may control the frequency of the reciprocating sliding to include a period in which the frequency becomes constant, while the frequency of the reciprocating sliding changes with time. Such an anomalous rhythm can also relax the user and lead the user to sleep comfortably.

(7) The control means may control the frequency of the reciprocating sliding based on the biometric information of the bed user detected by a sensor. By taking the biometric information of the bed user into account, it is possible to give oscillation according to the state of the user.

(8) The biological information may be, for example, an electroencephalogram, a respiration rate, a heart rate, a body temperature, or a combination thereof.

### BRIEF DESCRIPTION OF THE DRAWINGS

FIG. 1 is a view showing an example of the configuration of a sleep induction device 100 of the present invention.

FIGS. 2A to 2D are views showing the operation principle of the movable leg holder 113 by the drive means 114 employing the crank mechanism.

FIGS. 3A to 3C show the type of leg 12 of bed 10.

FIGS. 4A to 4C are views showing states in which the bed 10 is mounted on the drive leg support 110 and the driven leg support 120.

FIGS. 5A to 5C are views showing states in which the bed 10 is moved by the movement of the drive leg support 110.

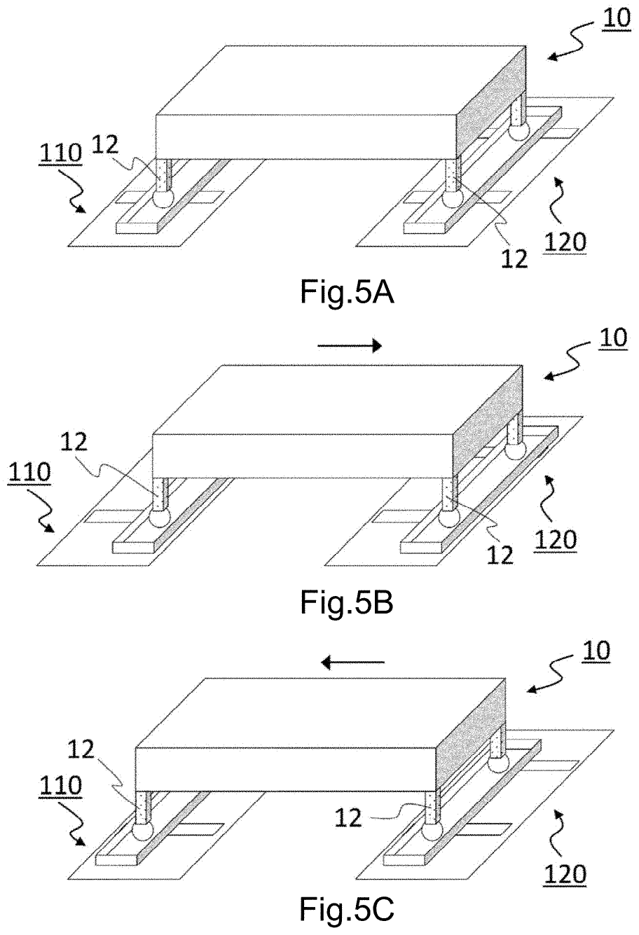

### DESCRIPTION OF EMBODIMENTS

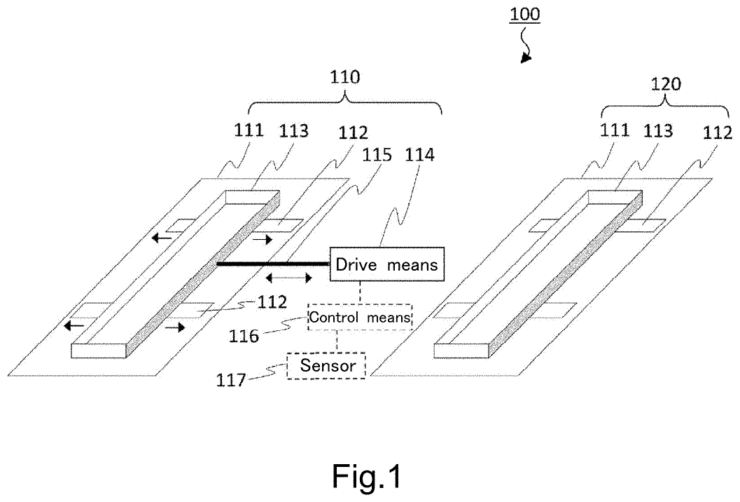

FIG. 1 shows an example of a configuration of a sleep induction device 100 according to the present invention. The sleep induction device 100 includes a drive leg support 110 including a base 111, two rail guides 112, a movable leg holder 113, and a drive means 114, and a driven leg support 120 including a base 111, two rail guides 112, and a movable leg holder 113.

In the drive leg support 110 and the driven leg support 120, the two rail guides 112 are fixed to the base 111 so that their longitudinal directions are parallel to each other. The two rail guides 112 are provided so that the movable leg holder 113 can freely slide in the longitudinal direction of the two rail guides 112 within the range of the length of the rail guides 112. That is, the length of the rail guide 112 needs to be longer than the range in which the movable leg holder 113 is to be slid.

In the drive leg support 120, a drive means 114 for driving the movable leg holder 113 to reciprocate within the range of the length of the rail guide 112 is connected via a connecting rod 115.

The method in which the drive means 114 drives the movable leg holder 113 to reciprocate within the length of the rail guide 112 is arbitrary. As an easy method, for example, a method using a crank mechanism for converting a rotational motion by a motor into a reciprocating motion can be cited.

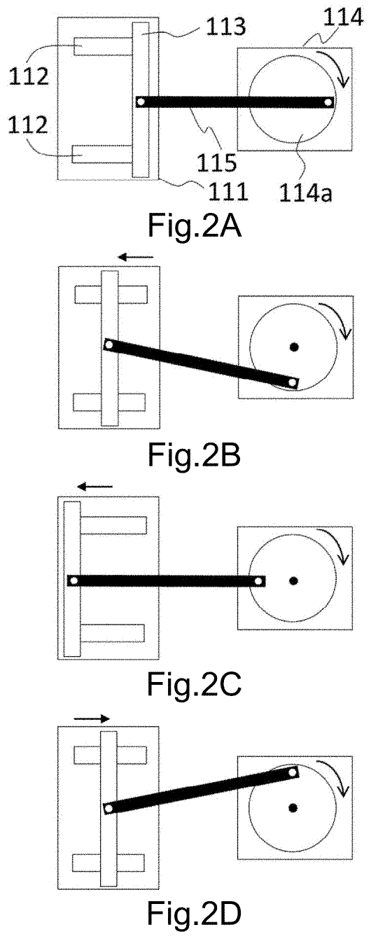

An outline of a method of reciprocating the movable leg holder 113 by the crank mechanism will be described with reference to FIGS. 2A to 2D. The drive means 114 includes a crank disk 114a which is rotated by a motor (not shown). One end of the connecting rod 115 is connected to the peripheral portion of the crank disk 114a, and the other end is connected to the movable leg holder 113. As shown in FIG. 2A, the positional relationship between the base 111 and the drive means 114 is determined so that the connection point between the connecting rod 115 and the crank disk 114a is located at a position farthest from the base 111 when the movable leg holder 113 is located closest to the drive means 114. As a result, when the crank disk 114a rotates clockwise, the movable leg holder 113 is provided so as to be freely slidable in the longitudinal direction of the rail guide 112, so that the movable leg holder 113 is gradually pushed in the longitudinal direction of the rail guide 112, and the movable leg holder 113 moves to the side farthest away from the drive means 114 as shown in FIG. 2C through the state shown in FIG. 2B. When the crank disk 114a further rotates clockwise, the movable leg holder 113 is gradually pulled in the longitudinal direction of the rail guide 112, and the state returns to the state of FIG. 2A through the state of FIG. 2A. In this manner, the rotational motion of the crank disk 114a can be converted into the reciprocating motion of the movable leg holder 113. When the drive means 114 is configured in this manner, the amplitude of the movable leg holder 113 can be adjusted by the diameter of the crank disk 114a.

The movable leg holder 113 provided in each of the drive leg support 110 and the driven leg support 120 is a leg holder for mounting the leg portion 11 of the bed 10 composed of the bed portion 11 and the leg portion 12.

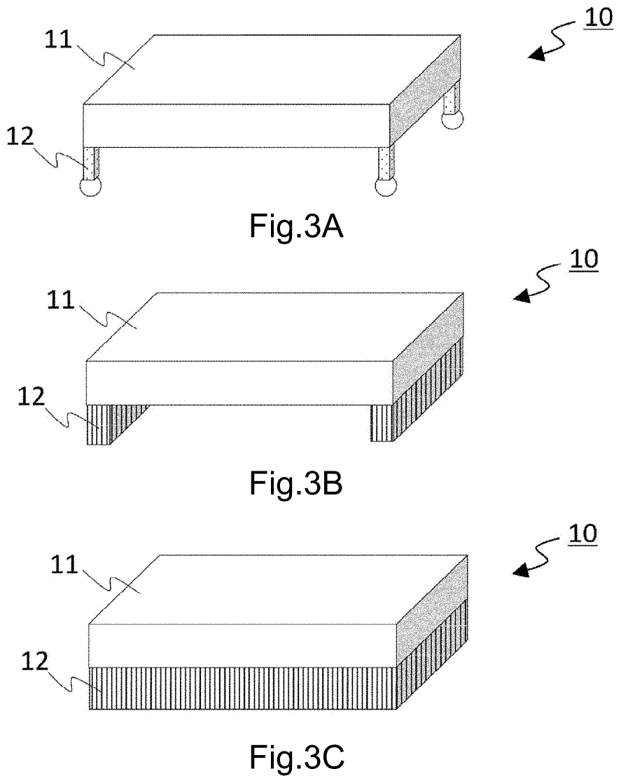

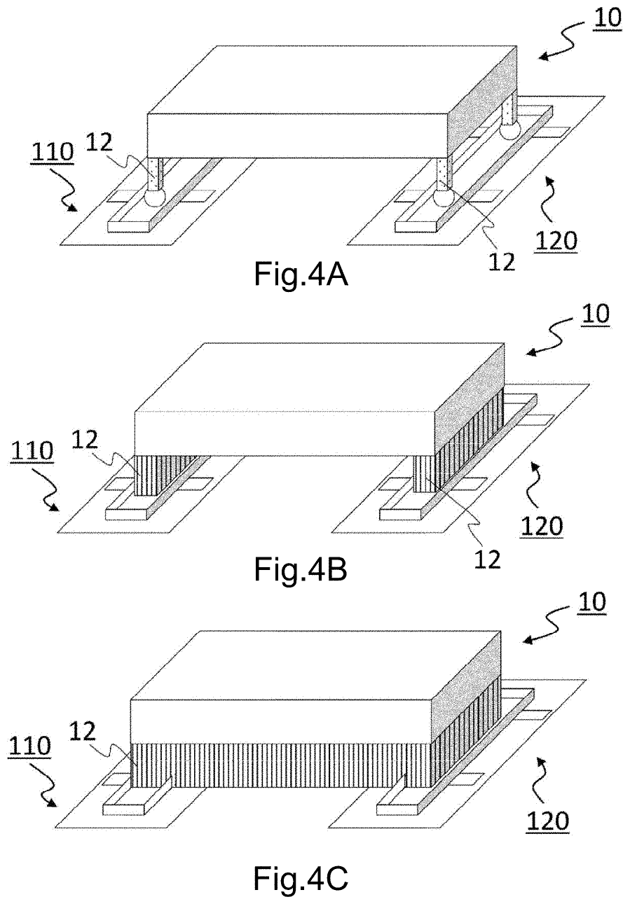

In general, the leg portion 12 of the bed 10 includes those for supporting the bed portion 11 at four corners as shown in FIG. 3A, those for supporting the bed portion 11 at two sides as shown in FIG. 3B, and those for supporting the entire periphery of the bed portion 11 as shown in FIG. 3C. The sleep induction device 100 of the present invention can be applied to any of these leg-shaped beds. For example, when the movable leg holder 113 has the shape shown in FIG. 1, the leg portion 12 on one end side in the longitudinal direction of the bed 10 in FIGS. 3A and 3B can be mounted on the movable leg holder 113 of the drive leg support 110, and the leg portion 12 on the other end side can be mounted on the movable leg holder 113 of the driven leg support 120, as shown in FIGS. 4A and 4B, respectively. The leg portion 12 of the bed 10 shown in FIG. 3C can be placed by providing notched portions in the movable leg holders 113 as shown in FIG. 4C, for example.

The shape of the movable leg holder 113 shown in FIGS. 1 and 4 is an example, and any shape corresponding to the shape of the leg 12 may be used as long as the leg 12 of the bed 10 can be stably gripped against oscillation.

When the leg portion of the bed 10 is mounted on the movable leg holder 113 of the drive leg support 110 and the movable leg holder 113 of the driven leg support 120, the drive leg support 110 and the driven leg support 120 are installed so that the direction in which the movable leg holder 113 of the drive leg support 110 can slide matches the direction in which the movable leg holder 113 of the driven leg support 120 can slide. As a result, when the drive means 114 reciprocates and slides the movable leg holder 113 of the drive leg support 110, the movable leg holder 113 of the driven leg support 120 also reciprocates and slides, and thus the bed 10 can be oscillated.

Since the leg holders on which the bed 10 is placed is separated into the movable leg holder 110 and the driven leg holder 120, the above described sleep induction device 100 of the present invention can be applied regardless of the length of the bed 10 in the longitudinal direction. Also, in the lateral direction of the bed 10, the length of the movable leg holder 113 in the longitudinal direction is designed to be equal to or greater than the maximum width of the bed supposed to be placed, so that the bed can be applied to a bed of an arbitrary length within the range.

Further, in the sleep induction device 100 of the present invention, the bed 10 is oscillated in the longitudinal direction. This can suppress irritation to the semicircular canal of bed-users in the recumbent position and lead them to sleep comfortably.

Since the frequency of the oscillation that leads the bed user to sleep is not necessarily constant, the control means 116 for controlling the frequency of the reciprocating sliding of the drive means 114 may be provided so that the oscillation can be appropriately controlled.

When a crank mechanism is employed as the drive means 114, the frequency of reciprocating sliding can be controlled by controlling the rotational speed of the motor by the control means 116. The adoption of the crank mechanism creates an anomalous rhythm of decelerating, reversing, and accelerating before and after the turning portion of the reciprocating motion, and this rhythm can relax the user and lead the user to sleep comfortably.

The control means 116 may control the frequency of the reciprocating slide to vary with time. Such an anomalous rhythm can also relax the user and lead the user to sleep comfortably.

The control means 116 may control the frequency to include a period in which the frequency becomes constant, while the frequency of the reciprocating sliding changes with time. Such an anomalous rhythm can also relax the user and lead the user to sleep comfortably.

The control means 116 may control the frequency of the reciprocating sliding based on the biometric information of the bed user detected by the sensor 117. The biological information detected by the sensor 117 may be arbitrary, and may include, for example, electroencephalogram, respiration rate, heart rate, body temperature, or a combination thereof. By taking the biometric information of the bed user into account, it is possible to give oscillation according to the state of the user.

The present invention is not limited to the above embodiments. Each of the above embodiments is exemplary, and any of the embodiments having a substantially identical configuration and having a similar effect to the technical concept described in the claims of the present invention is included in the technical scope of the present invention.

### REFERENCE SIGNS LIST

10 Bed
11 Berth
12 Leg
100 Sleep induction device
110 Drive leg support
111 Base
112 Rail guide
113 Movable leg holder
114 Drive means
115 Connecting rod
116 Control means
117 Sensor
120 Driven leg support

1. A sleep induction device comprising:
a drive leg support including a movable leg holder slidable in a predetermined direction within a predetermined range, and drive means for reciprocally sliding the movable leg holder within the predetermined range,
a driven leg support provided with the movable leg holder,
wherein the sleep induction device oscillates a bed having legs mounted on the drive leg support and the driven leg support by reciprocally sliding the movable leg holder of the drive leg support.

2. The sleep induction device according to claim 1, wherein the predetermined direction is the longitudinal direction of the bed, and the leg portion on one longitudinal end side of the bed is mounted on the drive leg support, and the leg portion on the other end side is mounted on the driven leg support.

3. The sleep induction device according to claim 2, further comprising control means for controlling the frequency of reciprocating sliding of the drive means.

4. The sleep induction device according to claim 3, wherein the drive means realizes the reciprocating sliding by using a crank mechanism for converting the rotational motion by the motor into the reciprocating motion, and
the control means controls the frequency of the reciprocating slide by controlling the rotational speed of the motor.

5. The sleep induction device according to claim 3, wherein the control means controls so that the frequency of the reciprocating sliding changes with time.

6. The sleep induction device according to claim 5, wherein the control means controls the frequency of the reciprocating sliding so as to include a period in which the frequency becomes constant.

7. The sleep induction device according to claim 3, wherein the control means controls the frequency of the reciprocating sliding based on the biometric information of the user of the bed detected by a sensor.

8. The sleep induction device according to claim 7, wherein the biological information is one of brain waves, respiration rate, heart rate, and body temperature, or a combination thereof.
